(19) United States
(12) Patent Application Publication
(10) Pub. No.: US 2017/0105919 A1
(43) Pub. Date: Apr. 20, 2017

(54) HAIR CARE COMPOSITION COMPRISING ANTIDANDRUFF AGENT AND SILICONE

(71) Applicant: The Procter & Gamble Company, Cincinnati, OH (US)

(72) Inventor: Toshiyuki Iwata, Singapore (SG)

(21) Appl. No.: 15/293,709

(22) Filed: Oct. 14, 2016

Related U.S. Application Data
(60) Provisional application No. 62/241,147, filed on Oct. 14, 2015.

(51) Int. Cl. A61K 8/58; A61Q 5/12; A61K 8/898; A61Q 5/00; A61K 8/41; A61K 8/34

(52) U.S. Cl. CPC A61K 8/58; A61K 8/416; A61K 8/342; A61K 8/898; A61Q 5/006; A61Q 5/12; A61K 2800/524; A61K 2800/5922

(57) ABSTRACT

Disclosed is a hair care composition comprising: a metal pyrithione; and a silicone compound having a linear silicone backbone, an amine or a quaternary ammonium group, and an alkylene oxide group; and further comprising polyquaternium-6. The compositions of the present invention provide improved deposition of antidandruff agents such as zinc pyrithione on scalp.

### FIELD OF THE INVENTION

The present invention relates to a hair care composition comprising: a metal pyrithione; and a silicone compound having a linear silicone backbone, an amine or a quaternary ammonium group, and an alkylene oxide group; and further comprising polyquaternium-6. The compositions of the present invention provide improved deposition of antidandruff agents such as zinc pyrithione on scalp.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. Furthermore, a variety of approaches have been developed to provide other benefits in addition to such conditioning benefits.

For example, some conditioning compositions provide antidandruff benefit in addition to conditioning benefits.

German patent application No. DE 10 2011 086 635 A discloses a cosmetic composition containing at least one dandruff control agent such as zinc pyrithione, and at least one cationic aminosilicone with at least three terminal amino-functional groups. The compositions of this publication are said to have, in addition to conditioning effects and improvement of the shine and feel of keratinic fibers, particularly human hair, improved effects regarding reduction of formation of dandruff. The German publication also discloses such compositions in Examples containing 0.5% of zinc pyrithione, and 0.5% or 1.0% of silicone quaternium-22.

PCT patent publication No. WO 2014/014977 discloses a hair care composition comprising polyquaternium-6 and metal pyrithiones as antidandruff agents, wherein the composition is substantially free of glucan gum, nonionic guar gum and hydroxyethylcellulose. The composition of this publication are said to provides improved deposition of antidandruff agents such as zinc pyrithione on scalp. The WO publication also discloses such hair care composition in Example comprising 0.075% of polyquaternium-6, 0.75% of zinc pyrithione, 1.6% of zinc carbonate, and 2.0% of aminosilicone.

There still exists a need for such antidandruff hair care compositions, to provide improved deposition of antidandruff agents such as zinc pyrithione on scalp.

None of the existing art provides all of the advantages and benefits of the present invention.

### SUMMARY OF THE INVENTION

The present invention is directed to hair care composition comprising by weight:

(a) from about 0.1% to about 10% of a cationic surfactant system;
(b) from about 0.1% to about 20% of a high melting point fatty compound;
(c) from about 0.01% to about 5% of a metal pyrithione;
(d) from about 0.05% to about 15% of a silicone compound having: a linear silicone backbone; an amine or a quaternary ammonium group; and an alkylene oxide group;
(e) from about 0.01% to about 1% of polyquaternium-6; and
(f) an aqueous carrier.

The compositions of the present invention provide improved deposition of antidandruff agents such as zinc pyrithione on scalp.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

The term “molecular weight” or “M.Wt.” as used herein refers to the weight average molecular weight unless otherwise stated.

### Composition

The composition comprises a cationic surfactant system; a high melting point fatty compound; a metal pyrithione; silicone compound having a linear silicone backbone, an amine or a quaternary ammonium group, and an alkylene oxide group; and an aqueous carrier, and further comprising polyquaternium-6. It is believed that the silicone compound having a linear silicone backbone provide improved deposition of metal pyrithione when used in the composition of the present invention containing Polyquaternium-6, compared to similar silicone compounds having a branched silicone backbone such as Silicone Quaternium-22.

These ingredients, as well as the gel matrix formed by some of these ingredients, are explained below in detail.

The composition of the present invention is, preferably, substantially free of anionic surfactants in view of avoiding undesirable interaction with cationic surfactants and/or in view of stability of the gel matrix. In the present invention, “the composition being substantially free of anionic surfactants” means that: the composition is free of anionic surfactants; or, if the composition contains anionic surfactants, the level of such anionic surfactants is very low. In the present invention, the total level of such anionic surfactants is, if included, 1% or less, preferably 0.5% or less, more preferably 0.1% or less, still more preferably 0% by weight of the composition.

### Cationic Surfactant System

The composition of the present invention comprises a cationic surfactant system. The cationic surfactant system can be one cationic surfactant or a mixture of two or more cationic surfactants. Preferably, the cationic surfactant system is selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt. More preferably, the cationic surfactant system is a mixture of mono-long alkyl quaternized ammonium salt and di-long alkyl quaternized ammonium salt.

The cationic surfactant system is included in the composition at a level by weight of preferably from about 0.1% to about 10%, more preferably from about 0.5% to about 8%, still more preferably from about 0.8% to about 5%, even more preferably from about 1.0% to about 4%.

### Mono-Long Alkyl Quaternized Ammonium Salt

The monoalkyl quaternized ammonium salt cationic surfactants useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

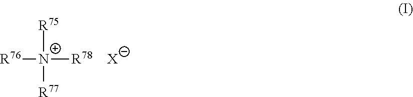

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

### Di-Long Alkyl Quaternized Ammonium Salt

Di-long alkyl quaternized ammonium salt is preferably combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of preferably from about 10% to about 50%, more preferably from about 30% to about 45%.

The dialkyl quaternized ammonium salt cationic surfactants useful herein are those having two long alkyl chains having 12-30 carbon atoms, preferably 16-24 carbon atoms, more preferably 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

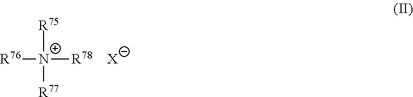

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### High Melting Point Fatty Compound

The composition of the present invention comprises a high melting point fatty compound. The high melting point fatty compound is included in the composition at a level of preferably from about 0.1% to about 20%, more preferably from about 1% to about 15%, still more preferably from about 1.5% to about 8% by weight of the composition, in view of providing improved conditioning benefits such as slippery feel during the application to wet hair, softness and moisturized feel on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

### Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

### Gel Matrix

The composition of the present invention comprises a gel matrix. The gel matrix comprises a cationic surfactant, a high melting point fatty compound, and an aqueous carrier.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

### Silicone Compound

The compositions of the present invention comprise a silicone compound having: a linear silicone backbone; an amine or a quaternary ammonium group; and an alkylene oxide group. The silicone compound is contained in the composition at a level of from about 0.05% to about 15%, preferably from about 0.1% to about 10%, more preferably from about 0.15% to about 5%, and even more preferably from about 0.2% to about 4% by weight of the composition.

Such silicone compounds having: a linear silicone backbone; an amine or a quaternary ammonium group; and an alkylene oxide group are, for example, those described below in “Silicone Polymer Containing Quaternary Ammonium Group”.

It is believed that the silicone compound having a linear silicone backbone provide improved deposition of metal pyrithione when used in the composition of the present invention containing Polyquaternium-6, compared to similar silicone compounds having a branched silicone backbone such as Silicone Quaternium-22.

More preferably for providing improved deposition of metal pyrithione, in the silicone compounds useful herein, the alkylene oxide groups are located in a silicone backbone of the silicone compound, rather than grafting to the backbone as pendant groups. Still more preferably for providing improved deposition of metal pyrithione, the amine or quaternary ammonium groups are also located in the backbone of the silicone compound, rather than grafting to the backbone as pendant groups. Such highly preferred silicone compounds are explained below in detail.

### Silicone Polymer Containing Quaternary Ammonium Groups

Silicone compounds useful herein include, for example, a Silicone Polymer Containing Quaternary Groups comprising terminal ester groups, having a viscosity up to 100,000 mPa·s and a D block length of greater than 200 D units. Without being bound by theory, this low viscosity silicone polymer provides improved conditioning benefits such as smooth feel, reduced friction, and prevention of hair damage, while eliminating the need for a silicone blend.

Structurally, the silicone polymer is a polyorganosiloxane compound comprising one or more quaternary ammonium groups, at least one silicone block comprising greater than 200 siloxane units, at least one polyalkylene oxide structural unit, and at least one terminal ester group. In one or more embodiments, the silicone block may comprise between 300 to 500 siloxane units.

In a preferred embodiment, the polyorganosiloxane compounds have the general formulas (Ia) and (Ib):

M-Y—[—(N⁺R₂-T-N⁺R₂)—Y—]ₘ—[—(NR²-A-E-A′-NR²)—Y-]ₖ-M (Ia)

M-Y—[—(N⁺R₂-T-N⁺R₂)—Y—]ₘ—[—(N⁺R²₂-A-E-A′-N⁺R²₂)—Y-]ₖ-M (Ib)

wherein:
m is >0, preferred 0.01 to 100, more preferred 0.1 to 100, even more preferred 1 to 100, specifically 1 to 50, more specifically 1 to 20, even more specifically 1 to 10,
k is 0 or an average value of from >0 to 50, or preferably from 1 to 20, or even more preferably from 1 to 10,
M represents a terminal group, comprising terminal ester groups selected from

—OC(O)—Z

—OS(O)₂—Z

—OS(O₂)O—Z

—OP(O)(O—Z)OH

—OP(O)(O—Z)₂

wherein Z is selected from monovalent organic residues having up to 40 carbon atoms, optionally comprising one or more hetero atoms.
A and A′ each are independently from each other selected from a single bond or a divalent organic group having up to 10 carbon atoms and one or more hetero atoms, and
E is a polyalkylene oxide group of the general formula:

—[CH₂CH₂O]_{q}—[CH₂CH(CH₃)O]ᵣ—[CH₂CH(C₂H₅)O]ₛ—

wherein q=0 to 200, r=0 to 200, s=0 to 200, and q+r+s=1 to 600.
R² is selected from hydrogen or R,
R is selected from monovalent organic groups having up to 22 carbon atoms and optionally one or more heteroatoms, and wherein the free valencies at the nitrogen atoms are bound to carbon atoms,
Y is a group of the formula:

—K—S—K— and -A-E-A′- or -A′-E-A-,

with S=

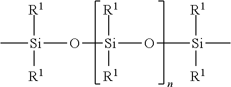

wherein R1═C₁-C₂₂-alkyl, C₁-C₂₂-fluoralkyl or aryl; n=200 to 1000, and these can be identical or different if several S Groups are present in the polyorganosiloxane compound.
K is a bivalent or trivalent straight chain, cyclic and/or branched C₂-C₄₀ hydrocarbon residue which is optionally interrupted by —O—, —NH—, trivalent N, —NR¹—, —C(O)—, —C(S)—, and optionally substituted with —OH, wherein R¹ is defined as above,
T is selected from a divalent organic group having up to 20 carbon atoms and one or more hetero atoms.

The residues K may be identical or different from each other. In the —K—S—K— moiety, the residue K is bound to the silicon atom of the residue S via a C—Si-bond.

Due to the possible presence of amine groups (—(NR²-A-E-A′-NR²)—) in the polyorganosiloxane compounds, they may have protonated ammonium groups, resulting from the protonation of such amine groups with organic or inorganic acids. Such compounds are sometimes referred to as acid addition salts of the polyorganosiloxane compounds.

In a preferred embodiment the molar ratio of the quaternary ammonium groups b) and the terminal ester groups c) is less than 100:20, even more preferred is less than 100:30 and is most preferred less than 100:50. The ratio can be determined by ¹³C-NMR.

In a further embodiment, the polyorganosiloxane composition may comprise:

A) at least one polyorganosiloxane compound, comprising a) at least one polyorganosiloxane group, b) at least one quaternary ammonium group, c) at least one terminal ester group, and d) at least one polyalkylene oxide group (as defined before),
B) at least one polyorganosiloxane compound, comprising at least one terminal ester group, different from compound A).

In the definition of component A) it can be referred to the description of the polyorganosiloxane compounds of the invention. The polyorganosiloxane compound B) differs from the polyorganosiloxane compound A) preferably in that it does not comprise quaternary ammonium groups. Preferred polyorganosiloxane compounds B) result from the reaction of monofunctional organic acids, in particular carboxylic acids, and polyorganosiloxane containing bisepoxides.

In the polyorganosiloxane compositions the weight ratio of compound A) to compound B) is preferably less than 90:10. Or in other words, the content of component B) is at least 10 weight percent. In a further preferred embodiment of the polyorganosiloxane compositions in compound A) the molar ratio of the quaternary ammonium groups b) and the terminal ester groups c) is less than 100:10, even more preferred is less than 100:15 and is most preferred less than 100:20.

The silicone polymer has a viscosity at 20° C. and a shear rate of 0.1 s⁻¹ (plate-plate system, plate diameter 40 mm, gap width 0.5 mm) of less than 100,000 mPa·s (100 Pa·s). In further embodiments, the viscosities of the neat silicone polymers may range from 500 to 100,000 mPa·s, or preferably from 500 to 70,000 mPa·s, or more preferably from 500 to 50,000 mPa·s, or even more preferably from 500 to 20,000 mPa·s. In further embodiments, the viscosities of the neat polymers may range from 500 to 10,000 mPa·s, or preferably 500 to 5000 mPa·s determined at 20° C. and a shear rate of 0.1 s⁻¹.

In addition to the above listed silicone polymers, the following preferred compositions are provided below. For example, in the polyalkylene oxide group E of the general formula:

—[CH₂CH₂O]_{q}—[CH₂CH(CH₃)O]ᵣ—[CH₂CH(C₂H₅)O]ₛ—

wherein the q, r, and s indices may be defined as follows:
q=0 to 200, or preferably from 0 to 100, or more preferably from 0 to 50, or even more preferably from 0 to 20,
r=0 to 200, or preferably from 0 to 100, or more preferably from 0 to 50, or even more preferably from 0 to 20,
s=0 to 200, or preferably from 0 to 100, or more preferably from 0 to 50, or even more preferably from 0 to 20,
and q+r+s=1 to 600, or preferably from 1 to 100, or more preferably from 1 to 50, or even more preferably from 1 to 40.

For polyorganosiloxane structural units with the general formula S:

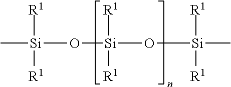

R¹═C₁-C₂₂-alkyl, C₁-C₂₂-fluoralkyl or aryl; n=from 200 to 1000, or preferably from 300 to 500, K (in the group —K—S—K—) is preferably a bivalent or trivalent straight chain, cyclical or branched C₂-C₂₀ hydrocarbon residue which is optionally interrupted by —O—, —NH—, trivalent N, —NR¹—, —C(O)—, —C(S)—, and optionally substituted with —OH.

In specific embodiments, R¹ is C₁-C₁₈ alkyl, C₁-C₁₈ fluoroalkyl and aryl. Furthermore, R¹ is preferably C₁-C₁₈ alkyl, C₁-C₆ fluoroalkyl and aryl. Furthermore, R¹ is more preferably C₁-C₆ alkyl, C₁-C₆ fluoroalkyl, even more preferably C₁-C₄ fluoroalkyl, and phenyl. Most preferably, R¹ is methyl, ethyl, trifluoropropyl and phenyl.

As used herein, the term “C₁-C₂₂ alkyl” means that the aliphatic hydrocarbon groups possess from 1 to 22 carbon atoms which can be straight chain or branched. Methyl, ethyl, propyl, n-butyl, pentyl, hexyl, heptyl, nonyl, decyl, undecyl, isopropyl, neopentyl and 1,2,3-trimethyl hexyl moieties serve as examples.

Further as used herein, the term “C₁-C₂₂ fluoroalkyl” means aliphatic hydrocarbon compounds with 1 to 22 carbon atoms which can be straight chain or branched and are substituted with at least one fluorine atom. Monofluormethyl, monofluoroethyl, 1,1,1-trifluorethyl, perfluoroethyl, 1,1,1-trifluoropropyl, 1,2,2-trifluorobutyl are suitable examples.

Moreover, the term “aryl” means unsubstituted or phenyl substituted once or several times with OH, F, Cl, CF₃, C₁-C₆ alkyl, C₁-C₆ alkoxy, C₃-C₇ cycloalkyl, C₂-C₆ alkenyl or phenyl. Aryl may also mean naphthyl.

For the embodiments of the polyorganosiloxanes, the positive charges resulting from the ammonium group(s), are neutralized with inorganic anions such as chloride, bromide, hydrogen sulfate, sulfate, or organic anions, like carboxylates deriving from C₁-C₃₀ carboxylic acids, for example acetate, propionate, octanoate, especially from C₁₀-C₁₈ carboxylic acids, for example decanoate, dodecanoate, tetradecanoate, hexadecanoate, octadecanoate and oleate, alkylpolyethercarboxylate, alkylsulphonate, arylsulphonate, alkylarylsulphonate, alkylsulphate, alkylpolyethersulphate, phosphates derived from phosphoric acid mono alkyl/aryl ester and phosphoric acid dialkyl/aryl ester. The properties of the polyorganosiloxane compounds can be, inter alia, modified based upon the selection of acids used.

The quaternary ammonium groups are usually generated by reacting the di-tertiary amines with an alkylating agents, selected from in particular di-epoxides (sometimes referred to also as bis-epoxides) in the presence of mono carboxylic acids and difunctional dihalogen alkyl compounds.

In a preferred embodiment the polyorganosiloxane compounds are of the general formulas (Ia) and (Ib):

M-Y—[—(N⁺R₂-T-N⁺R₂)—Y—]ₘ—[—(NR²-A-E-A′-NR²)—Y—]ₖ-M (Ia)

M-Y—[—(N⁺R₂-T-N⁺R₂)—Y—]ₘ—[—(N⁺R²₂-A-E-A′-N⁺R²₂)—Y—]ₖ-M (Ib)

wherein each group is as defined above; however, the repeating units are in a statistical arrangement (i.e., not a block-wise arrangement).

In a further preferred embodiment the polyorganosiloxane compounds may be also of the general formulas (IIa) or (IIb):

M-Y—[—N⁺R₂—Y—]ₘ—[—(NR²-A-E-A′-NR²)—Y—]ₖ-M (IIa)

M-Y—[—N⁺R₂—Y—]ₘ—[—(N⁺R²₂-A-E-A′-N⁺R²₂)—Y—]ₖ-M (IIb)

wherein each group is as defined above. Also in such formula the repeating units are usually in a statistical arrangement (i.e not a block-wise arrangement).
wherein, as defined above, M is

—OC(O)—Z,

—OS(O)₂—Z

—OS(O₂)O—Z

—OP(O)(O—Z)OH

—OP(O)(O—Z)₂

Z is a straight chain, cyclic or branched saturated or unsaturated C₁-C₂₀, or preferably C₂ to C₁₈, or even more preferably a hydrocarbon radical, which can be interrupted by one or more —O—, or —C(O)— and substituted with —OH. In a specific embodiment, M is —OC(O)—Z resulting from normal carboxylic acids in particular with more than 10 carbon atoms like for example dodecanoic acid.

In a further embodiment, the molar ratio of the polyorganosiloxane-containing repeating group —K—S—K— and the polyalkylene repeating group -A-E-A′- or -A′-E-A- is between 100:1 and 1:100, or preferably between 20:1 and 1:20, or more preferably between 10:1 and 1:10.

In the group —(N⁺R₂-T-N⁺R₂)—, R may represent a monovalent straight chain, cyclic or branched C₁-C₂₀ hydrocarbon radical, which can be interrupted by one or more —O—, —C(O)—and can be substituted by —OH, T may represent a divalent straight-chain, cyclic, or branched C₁-C₂₀ hydrocarbon radical, which can be interrupted by —O—, —C(O)— and can be substituted by hydroxyl.

The above described polyorganosiloxane compounds comprising quaternary ammonium functions and ester functions may also contain: 1) individual molecules which contain quaternary ammonium functions and no ester functions; 2) molecules which contain quaternary ammonium functions and ester functions; and 3) molecules which contain ester functions and no quaternary ammonium functions. While not limited to structure, the above described polyorganosiloxane compounds comprising quaternary ammonium functions and ester functions are to be understood as mixtures of molecules comprising a certain averaged amount and ratio of both moieties.

Various monofunctional organic acids may be utilized to yield the esters. Exemplary embodiments include C₁-C₃₀ carboxylic acids, for example C₂, C₃, C₈ acids, C₁₀-C₁₈ carboxylic acids, for example C₁₂, C₁₄, C₁₆ acids, saturated, unsaturated and hydroxyl functionalized C₁₈ acids, alkylpolyethercarboxylic acids, alkylsulphonic acids, arylsulphonic acids, alkylarylsulphonic acids, alkylsulphuric acids, alkylpolyethersulphuric acids, phosphoric acid mono alkyl/aryl esters and phosphoric acid dialkyl/aryl esters.

### Other Silicones

The compositions of the present invention may additionally contain other silicones than the above described silicone compounds, for example, at a level of preferably from about 0.05% to about 10%, more preferably from about 0.1% to about 5%.

Such other silicones can be, for example, volatile silicones such as cyclic silicones, dimethylpolysiloxane fluid, dimethylpolysiloxane gum, amino silicone, and silicone copolyol. Preferred aminosilicones include, for example, those which conform to the general formula (I):

(R₁)ₐG₃₋ₐ-Si—(—OSiG₂)ₙ-(—OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

### Metal Pyrithione

The compositions of the present invention comprise a metal pyrithione. The metal pyrithione can be included at a level by weight of the compositions of, preferably from about 0.01% to about 5%, more preferably from about 0.1% to about 3%, still more preferably from about 0.1% to about 2%.

In one embodiment of the present invention, the weight ratio of the metal pyrithione to the silicone compound is preferably from about 4:1 to about 1:40, more preferably from about 2:1 to about 1:40, still more preferably from about 1:1 to about 1:40, even more preferably from about 1:1 to about 1:20, further preferably from about 1:1.5 to about 1:10. It is believed that the above specific weight ratio, together with the specific silicone compound, provides improved deposition of the metal pyrithione.

Metal pyrithiones useful herein are heavy metal salts of 1-hydroxy-2-pyridinethione, the heavy metal salts being zinc, tin, cadmium, magnesium, aluminium, barium, bismuth, strontium, copper, and zirconium. Preferred are zinc and copper. More preferred is zinc salt of 1-hydroxy-2-pyridinethione known in the art as zinc pyrithione, more preferably in a particle size of up to about 20 microns, still preferably from about 1 to about 10 microns.

### Polyquaternium-6

The composition comprises polyquaternium-6, i.e., homopolymer of diallyldimethylammonium chloride. It is believed that polyquaternium-6, together with the specific silicone compound, provides improved deposition of the metal pyrithione.

The polyquaternium-6 can be included in the composition at a level by weight of from about 0.001% to about 5%, preferably from about 0.01% to about 1%, more preferably from about 0.02% to about 0.5%, still more preferably from about 0.03% to about 0.3%, in view of providing improved deposition of metal pyrithione, and also in view of providing improved conditioning benefit by avoiding stickiness, hair clumping and/or build up which may cause when adding polyquaternium-6 at higher levels.

When included, the weight ratio of polyquaternium-6 to the metal pyrithione is preferably from about 1:1 to about 1:30 more preferably from about 1:2 to about 1:20, still more preferably from about 1:5 to about 1:15 in view of providing improved deposition of metal pyrithione while avoiding stickiness, hair clumping and/or build up.

The polyquaternium-6 useful herein is that having a cationic charge density of, preferably from about 3.5 meq/g, more preferably from about 4.5 meq/g, still more preferably from about 5.5 meq/g in view of providing improved deposition of metal pyrithione, and preferably to about 13 meq/g, more preferably to about 10 meq/g, still more preferably to about 7.0 meq/g, in view of providing improved deposition of the metal pyrithione.

The polyquaternium-6 useful herein is that having a molecular weight of, preferably about 800 g/mol or more, more preferably 1,000 g/mol or more, still more preferably 1,200 g/mol or more in view of providing improved deposition of metal pyrithione. The molecular weight is also preferably to about 1,000,000 g/mol, more preferably to about 500,000 g/mol, still more preferably to about 100,000 g/mol, even more preferably to about 50,000 g/mol in view of providing better conditioning while providing improved deposition of metal pyrithione.

Commercially available examples of highly preferred polyquaternium-6 polymer include, for example, that having a tradename Merquat 100 available from Lubrizol (acquisition), which has a cationic charge density of about 6.19 meq/g, molecular weight of about 150,000 g/mol, and that having a tradename Merquat 106 available from Lubrizol, which has a cationic charge density of about 6.19 meq/g, molecular weight of about 15,000 g/mol.

Metal Salt Other than Metal Pyrithione

In one embodiment of the present invention, the composition preferably comprises a metal salt other than metal pyrithiones. The metal salt is believed to improve antidandruff efficacy of metal pyrithione and/or hair volume benefit. It is also believed that the metal salt, together with the specific silicone compound, provides improved deposition of the metal pyrithione. The metal salt can be used at levels by weight of the composition of preferably from about 0.05% to about 10%, more preferably from about 0.1% to about 7%, still more preferably from about 0.5% to about 5% in view of delivering the above benefits.

When containing a metal pyrithione in the composition, metal salt is preferably, a salt of a metal which is the same metal as that of the metal pyrithione. For example, when the metal pyrithione is zinc pyrithione, the metal salt is preferably zinc salt other than zinc pyrithione. Such zinc salts include, for example, Zinc aluminate, Zinc carbonate, Zinc oxide, Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide, zinc hydroxyl carbonate, hydrozincite (zinc carbonate hydroxide), basic zinc carbonate, aurichalcite (zinc copper carbonate hydroxide), rosasite (copper zinc carbonate hydroxide) and combinations thereof. Preferably, zinc salt are zinc hydroxyl carbonate, hydrozincite (zinc carbonate hydroxide), basic zinc carbonate, aurichalcite (zinc copper carbonate hydroxide), rosasite (copper zinc carbonate hydroxide) and combinations thereof. More preferably, hydrozincite (zinc carbonate hydroxide) is used.

Preferably, such metal salt are those which remain mostly insoluble within formulated compositions. “Being insoluble within the formulated compositions” herein means that the material remains as solid particulates and do not dissolve in the formula

D(90) is the particle size which corresponds to 90% of the amount of particles are below this size. The particulate of metal salt preferably have a particle size distribution wherein 90% of the particles are less than about 50 microns. In a further embodiment of the present invention, the particulate metal salt may have a particle size distribution wherein 90% of the particles are less than about 30 microns. In yet a further embodiment of the present invention, the particulate metal salt may have a particle size distribution wherein 90% of the particles are less than about 20 microns.

### Anionic Polymer

The composition of the present invention may further contain an anionic polymer for the metal pyrithione. The anionic polymer can be used at levels by weight of the composition of preferably from about 0.001% to about 1%, more preferably from about 0.01% to about 0.80%, still more preferably from about 0.02% to about 0.6% in view of improving suspension of metal pyrithiones, improving deposition of metal pyrithiones especially when used together with Polyquaternium-6.

When included, it is preferred that the weight ratio of the anionic polymer to metal pyrithione, is from about 1:1 to about 1:100, more preferably from about 1:10 to about 1:50 still more preferably from about 1:20 to about 1:30, in view of improving suspension of metal pyrithiones,

Anionic polymers useful herein are, for example, those having a molecular weight of preferably from about 100 g/mol to about 100,000 g/mol more preferably from about 1,000 g/mol to about 10,000 g/mol still more preferably from about 1,000 g/mol to about 5,000 g/mol in comparison to standards of sodium poly(styrenesulfonate) in view of having the ability to suspend solids and prevent their agglomeration, and those having a charge density of from about 1.0 meq/g to about 10 meq/g more preferably from about 2.0 meq/g to about 7 meq/g still more preferably from about 3.0 meq/g to about 5.0 meq/g in view of compatibility with cationic materials and stability of the formula.

Anionic polymers useful herein include, for example, sodium polynaphthalene sulfonate, Sodium Lignosulfonate, sodium carboxymethyl cellulose, Sodium salt of hydrophobically modified maleic anhydride copolymer, Sodium polyacrylate, sodium polymethacrylate, ammonium polyacrylate, ammonium polymethacrylate, Sodium salt of polymethacrylic acid, preferably sodium polynaphthalene sulfonate, and sodium carboxymethyl cellulose, and more preferably sodium polynaphthalene sulfonate, still more preferably sodium polynaphthalene sulfonate having a tradename Darvanl Spray Dried, supplied from Vanderbilt Minerals having a molecular weight of about 3,000 g/mol in comparison to standards of sodium poly(styrenesulfonate) and a charge density of from about 3.5 to about 4.0 meq/g.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolyzed collagen with tradename Peptein 2000 available from Hormel, vitamin E with tradename Emix-d available from Eisai, panthenol available from Roche, panthenyl ethyl ether available from Roche, hydrolyzed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; and ultraviolet and infrared screening and absorbing agents such as octyl salicylate.

### Product Forms

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used by following steps:
(i) after shampooing hair, applying to the hair an effective amount of the conditioning compositions for conditioning the hair; and
(ii) then rinsing the hair.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Compositions (Wt %)

| | | Ex. | Ex. | Ex. | CEx. | CEx. |
|---|---|---|---|---|---|---|
| Components | | 1 | 2 | 3 | i | ii |
| Group O | Behenyl | 1.78 | 3.3 | — | 1.78 | 1.78 |
| | trimethylammonium | | | | | |
| | methosulfate | | | | | |
| | Behenyl | — | — | 2.28 | — | — |
| | trimethyl ammonium | | | | | |
| | chloride | | | | | |
| | Distearyl dimethyl | 0.64 | — | — | 0.64 | 0.64 |
| | ammonium chloride | | | | | |
| | Dicetyl | — | 0.95 | — | — | — |
| | dimethyl ammonium | | | | | |
| | chloride | | | | | |
| | Cetyl alcohol | 1.19 | 1.61 | 1.86 | 1.19 | 1.19 |
| | Stearyl alcohol | 2.94 | 4.04 | 4.64 | 2.94 | 2.94 |
| | Benzyl alcohol | 0.4 | 0.4 | 0.4 | 0.4 | 0.4 |
| Group W | Deionized Water | q.s. to 100% of the composition | | | | |
| | Preservative | — | — | — | — | 0.03 |
| | (Kathon CG) | | | | | |
| Group Z | Zinc pyrithione *1 | 0.75 | 0.75 | 0.75 | 0.75 | 0.50 |
| | Zinc carbonate *2 | 1.6 | 1.6 | 1.6 | 1.6 | — |
| | Polyquaternium-6 *3 | 0.075 | 0.075 | 0.075 | 0.075 | — |
| Group P/S | Silicone | 1.5 | 1.5 | 1.5 | — | 0.5 |
| | compound-1 *4 | | | | | |
| | Silicone | — | — | — | — | — |
| | compound-2 *5 | | | | | |
| | Aminosilicone *6 | — | — | — | 1.5 | — |
| | Perfume | 0.5 | 0.5 | 0.5 | 0.5 | — |
| | 6N HC1 | 0.05 | 0.05 | 0.05 | 0.05 | — |

| | | | | | |
|---|---|---|---|---|---|
| pH | 6.8-8.3 | 6.8-8.3 | 6.8-8.3 | 6.8-8.3 | 4.0-6.0 |
| Deposition of Zinc pyrithione | A1 | — | — | C1 | D1 |

### Compositions (Wt %)

| | | Ex. | Ex. | CEx. | CEx. |
|---|---|---|---|---|---|
| Components | | 4 | 5 | iii | iv |
| Group O | Behenyl | 1.78 | 1.78 | 1.78 | 1.78 |
| | trimethylammonium | | | | |
| | methosulfate | | | | |
| | Behenyl | — | — | — | — |
| | trimethyl ammonium | | | | |
| | chloride | | | | |
| | Distearyl dimethyl | — | — | — | — |
| | ammonium chloride | | | | |
| | Dicetyl | 0.51 | 0.51 | 0.51 | 0.51 |
| | dimethyl ammonium | | | | |
| | chloride | | | | |
| | Cetyl alcohol | 1.17 | 1.17 | 1.17 | 1.17 |
| | Stearyl alcohol | 2.94 | 2.94 | 2.94 | 2.94 |
| | Benzyl alcohol | 0.4 | 0.4 | 0.4 | 0.4 |
| Group W | Deionized Water | q.s. to 100% of the composition | | | |
| | Preservative | 0.03 | 0.03 | 0.03 | 0.03 |
| | (Kathon CG) | | | | |
| Group Z | Zinc pyrithione *1 | 0.75 | 0.75 | 0.75 | 0.75 |
| | Zinc carbonate *2 | — | — | — | — |
| | Polyquaternium-6 *3 | 0.075 | 0.8 | 0.075 | — |
| Group P/S | Silicone | 1.5 | 1.5 | — | 1.5 |
| | compound-1 *4 | | | | |
| | Silicone | — | — | 1.5 | — |
| | compound-2 *5 | | | | |
| | Aminosilicone *6 | — | — | — | — |
| | Perfume | 0.5 | 0.5 | 0.5 | 0.5 |
| | 6N HCl | — | — | — | — |

| | | | | |
|---|---|---|---|---|
| pH | 4.0-6.0 | 4.0-6.0 | 4.0-6.0 | 4.0-6.0 |
| Deposition of Zinc pyrithione | A2 | SSS2 | C2+ | C2 |

### Definitions of Components

*1 Zinc pyrithione: having a particle size of from about 1 to about 10 microns
*2 Zinc carbonate: having a particle size of from about 1 to about 10 microns
*3 Polyquaternium-6: Poly(diallyldimethylammonium chloride) supplied with a tradename Merquat 106 from Lubrizol having a charge density of about 6.2 meq/g, and molecular weight of about 15,000 g/mol
*4 Silicone compound-1: Available from Momentive having the following formula:

M-Y—[—(N⁺R₂-T-N⁺R₂)—Y—]ₘ—[—(N⁺R²₂-A-E-A′-N⁺R²₂)—Y-]ₖ-M

wherein

| | |
|---|---|
| M | lauric ester |
| Y | K—S—K |
| K | CH₂—CHOH—CH₂—O—C₃H₆ |
| S | PDMS block with 368 siloxane units |
| R, R² | Methyl |
| T | C₆H₁₂ |
| A | CH₂—COO— |
| A′ | CO—CH₂ |
| E | Ethylene oxide (CH₂—CH₂—O) |
| | with average degree of |
| | ethoxylation of 2 |
| Ratio of silicone | 1:1 |
| blocks:alkylene | |
| oxide blocks | |
| Total Viscosity | 4700 mPa · s |

*5 Silicone compound-2: Silicone Quaternium-22
*6 Available from Momentive having a viscosity 10,000 mPa·s, and having following formula (I):

(R₁)ₐG₃₋ₐ-Si—(—OSiG₂)ₙ-(—OSiG_{b}(R₁)_{2-b})ₘ—O—SiG₃₋ₐ(R₁)ₐ (I)
wherein G is methyl; a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —NH₂

### Method of Preparation

The above hair care compositions of “Ex. 1” through “Ex. 5” of the present invention and “CEx. i” through “CEx.iv” as comparative examples can be prepared by any conventional method well known in the art. They are suitably made by the following method:

Group O components are mixed and heated to from about 66° C. to about 85° C. to form an oil phase. Separately, Group W components are mixed and heated to from about 20° C. to about 48° C. to form an aqueous phase. In Becomix® direct injection rotor-stator homogenizer, the oil phase is injected and it takes 0.2 second or less for the oils phase to reach to a high shear field having an energy density of from 1.0×10⁵ J/m³ to 1.0×10⁷ J/m³ where the aqueous phase is already present. Group P/S components are added to the gel matrix with agitation. If included, other components are added to the gel matrix with agitation. Then, Group Z components are added with agitation at a temperature of about 30° C. Then the composition is cooled down to room temperature.

### Properties and Benefits

For some of the compositions, some benefits are evaluated by the following methods. Results of the evaluation are shown above in Table.

Examples 1 through 5 are hair conditioning compositions of the present invention which are particularly useful for rinse-off use. The embodiments disclosed and represented by the previous “Ex. 1” through “Ex. 5” have many advantages. For example, they provide improved deposition of metal pyrithiones.

Such advantages can be understood by the comparison between the examples of the present inventionand comparative examples “CEx. i” and “CEx. iv”, especially when comparing “Ex.1” to comparative examples “CEx.i and CEx.ii”, and “Ex. 4 and Ex. 5” to “CEx. iii and CEx. iv”.

### Deposition Test

The deposition of the anti-dandruff active is measured by artificial skin substrates. First, a shampoo is applied to the artificial skin substrate, and washed away. Then, one of the compositions of the above examples is applied, and rinsed off. An aliquot of an extraction solution is added to the artificial skin substrate and agitated prior to recovery and analytical determination of zinc pyrithione content by conventional methodology, such as HPLC. The deposition amount of zinc pyrithione is evaluated as follows:

### Evaluation

SSS1 or SSS2: Above 200% (Excluding 200%) to 350% increased deposition, compared to Control (C1 or C2)
SS1 or SS2: Above 100% (Excluding 100%) to 200% increased deposition, compared to Control (C1 or C2)
S1 or S2: Above 40% (Excluding 40%) to 100% increased deposition, compared to Control (C1 or C2).
A1 or A2: Above 25% (Excluding 25%) to 40% increased deposition, compared to Control (C1 or C2).
B1 or B2: Above 10% (Excluding 10%) to 25% increased deposition, compared to Control (C1 or C2)
C1+ or C2+: Up to 10% increased deposition, compared to Control (C1 or C2). C1 or C2: Control
C1− or C2−: Up to 10% decreased deposition, compared to Control (C1 or C2).
D1 or D2: Above 10% (Excluding 10%) decreased deposition, compared to Control (C1 or C2)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising by weight:
(a) from about 0.1% to about 10% of a cationic surfactant system;
(b) from about 0.1% to about 20% of a high melting point fatty compound;
(c) from about 0.01% to about 5% of a metal pyrithione;
(d) from about 0.05% to about 15% of a silicone compound having: a linear silicone backbone; an amine or a quaternary ammonium group; and an alkylene oxide group;
(e) from about 0.01% to about 1% of polyquaternium-6; and
(f) an aqueous carrier.

2. The composition of claim 1, wherein the silicone compound has a quaternary ammonium group and an alkylene oxide group.

3. The composition of claim 2, wherein the silicone compound has: a quaternary ammonium group; and an alkylene oxide group located in the backbone of the silicone compound.
